(19) United States
(12) Patent Application Publication
(10) Pub. No.: US 2016/0113919 A1
(43) Pub. Date: Apr. 28, 2016

(54) CHLOROQUINE STEREOISOMER FOR TREATING TUBERCULOSIS RELATED DISEASES

(71) Applicant: The Board of Trustees of the Leland Stanford Junior University, Stanford, CA (US)

(72) Inventors: Niaz Banaei, Palo Alto, CA (US); Jeffrey S. Glenn, Palo Alto, CA (US); Rajiv Lochan Gaur, Palo Alto, CA (US); Tran Van, Stanford, CA (US); Gary K. Schoolnik, Stanford, CA (US); Edward Pham, Stanford, CA (US)

(21) Appl. No.: 14/918,404

(22) Filed: Oct. 20, 2015

Related U.S. Application Data
(60) Provisional application No. 62/067,056, filed on Oct. 22, 2014.

(51) Int. Cl. A61K 31/4706; A61K 45/06

(52) U.S. Cl. CPC A61K 31/4706; A61K 45/06

(57) ABSTRACT

This disclosure provides a method of treating a subject infected with a mycobacteria of the M. tuberculosis complex or infected with an atypical mycobacterium. Aspects of the method include administering to a subject in need thereof a therapeutically effective amount of an enantiomerically pure (R)-chloroquine agent. The (R)-chloroquine agent may be an analog or derivate of chloroquine having a particular stereochemistry at the position located alpha to the amino-quinoline core of the chloroquine agent. Also provided are methods of inhibiting mycobacteria of the M. tuberculosis complex or other atypical mycobacteria in a cell. Kits and pharmaceutical compositions for practicing the subject methods are also provided.

### GOVERNMENT SUPPORT

This invention was made with Government support under contracts RO1AI087917 and 1U19AI109662 awarded by the National Institutes of Health. The Government has certain rights in the invention.

### INTRODUCTION

Mycobacterium tuberculosis (TB) was responsible for 1.3 million deaths in 2012, second only to HIV/AIDS with respect to single infectious agents. TB is prevalent in the developing world, with 95% of TB deaths occurring in low and middle income countries. In addition, co-infection of TB and HIV is a significant health burden causing one fifth of deaths of HIV patients. Atypical mycobacteria are also responsible for significant disease burden.

TB is caused by the mycobacterium tuberculosis bacterium which commonly infects the lungs, but may also infect other parts of the body, including the kidney, the spine and the brain. If not treated effectively, TB can be fatal. TB may be transmitted through the air from an infected subject. Of great concern is multi-drug resistant strains of TB which are now present in most countries. Atypical mycobacteria, which include but are not limited to M. avium, M. intracellulare, M. abscessus ssp abscessus, M. abscessus ssp massiliense, M. kansasii, M. chelonae, M. xenopi, are also of increasing concern and for which current therapies are inadequate.

As such, compounds and methods useful in the treatment of TB and atypical mycobacteria, including drug resistant strains thereof, are of interest.

### SUMMARY

This disclosure provides a method of treating a subject infected with a mycobacteria of the M. tuberculosis complex, or infected with an atypical mycobacterium. Aspects of the method include administering to a subject in need thereof a therapeutically effective amount of an enantiomerically pure (R)-chloroquine agent. The (R)-chloroquine agent may be an analog or derivate of chloroquine having a particular stereochemistry at the position located alpha to the amino-quinoline core of the chloroquine agent. Also provided are methods of inhibiting mycobacteria of the M. tuberculosis complex, or other atypical mycobacteria in a cell.

Kits and pharmaceutical compositions for practicing the subject methods are also provided.

### BRIEF DESCRIPTION OF THE FIGURES

The skilled artisan will understand that the drawings, described below, are for illustration purposes only. The drawings are not intended to limit the scope of the present teachings in any way. It is emphasized that, according to common practice, the various features of the drawings are not to-scale. On the contrary, the dimensions of the various features are arbitrarily expanded or reduced for clarity. Included in the drawings are the following figures.

FIG. 1 illustrates that R-chloroquine (R-CQ) inhibits TB replication in macrophages more than S-chloroquine (S-CQ) or racemic chloroquine (CQ).

FIG. 2 illustrates that R-CQ is more active than S-CQ or racemic CQ against isoniazid (INH)-resistant TB in cells.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Furthermore, except as otherwise noted, the chemical methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. Enantiomeric and stereoisomeric mixtures can be resolved into their component enantiomers or stereoisomers using separation techniques or chiral synthesis techniques well known to the skilled artisan. In addition it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl(isopropyl), cyclopropan-1-yl, etc.; butanyls such as butan-1-yl, butan-2-yl(sec-butyl), 2-methyl-propan-1-yl(isobutyl), 2-methyl-propan-2-yl(t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl(allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

The term “aminoacyl” refers to the group —C(O)NR²¹R²², R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, .═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰(O⁻), —OP(O))(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

When the group being substituted is an aryl or heteroaryl group, the substituent(s) (e.g., as described herein) may be referred to as “aryl substituent(s)”.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

Numeric ranges are inclusive of the numbers defining the range.

The methods described herein include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

In further describing the subject invention, methods for treating or preventing infection are described first in greater detail. Next, enantiomerically pure chloroquine agents of interest for practicing the subject methods are reviewed. Methods of inhibiting mycobacteria of the M. tuberculosis complex, or atypical mycobacteria, in a cell are then described. Pharmaceutical compositions and kits are also described.

### Methods of Treating or Preventing Infection

As summarized above, aspects of the invention include methods of treating or preventing infection of a subject with a mycobacteria of the M. tuberculosis complex, or atypical mycobacteria. As such, aspects of the method include administering to a subject in need thereof a therapeutically effective amount of an enantiomerically pure chloroquine agent. In some instances, the agent is an enantiomerically pure (R)-chloroquine agent.

The term “mycobacteria of the M. tuberculosis complex” includes those species traditionally considered as causing the disease tuberculosis, as well as Mycobacterium environmental and opportunistic species that cause tuberculosis and lung disease in immune compromised patients, such as patients with AIDS, e.g., M. tuberculosis, M. bovis, or M. africanum, BCG, M. avium, M. intracellulare, M. celatum, M. genavense, M. haemophilum, M. kansasii, M. simiae, M. vaccae, M. fortuitum, and M. scrofulaceum (see, e.g., Harrison's Principles of Internal Medicine, Chapter 150, pp. 953-966 (16th ed., Braunwald, et al., eds., 2005). In certain embodiments, the mycobacteria is selected from M. tuberculosis, M. paratuberculosis, and M. leprae. In certain embodiments, the mycobacteria is M. tuberculosis.

The terms “atypical mycobacterium”, non-tuberculosis mycobacterium (NTM) or mycobacteria other than tuberculosis (MOTT) are synonomous. These microbes share many common properties, such as acid fastness and the ability to cause pulmonary and extrapulmonary granulomatous disorders. As a group, they comprise diverse organisms with dissimilarities in their cultural characteristics and pathogenicity to humans compared with M. tuberculosis (MTB). There are many organisms in this group capable of causing human infections. Infections caused by M. avium-intracellulare (MAI) complex became common in patients with severe HIV infection—in patients with low CD4 lymphocyte counts—or in AIDS, for which it is a disease-defining condition. NTM cause significant lung disease in individuals with structural abnormalities (e.g., patients with bronchiectasis and chronic obstructive pulmonary disease with M. kansasii infection) and in those with immunodeficiency syndromes (e.g., M. avium infection in HIV-AIDS patients).

The most common clinically important species in the slow growing group include the M. avium complex (M. avium and M. intracellulare), M. kansasii, M. xenopi, M. simiae, M. szulgai, M. scrofulaceum, M. malmoense, M. terrae-nonchromogenicum complex, M. haemophilum, and M. genavense. These organisms grow best at 35° to 37° C., with the exception of M. haemophilum, which has a preference for lower temperatures (28° to 30° C.) and the presence of iron, and M. xenopi, which grows optimally at 42° C. Newer isolated slow-growing species include M. celatum, M. interjectum, M. confluentis, M. triplex, M. lentiflavum, M. branderi, M. conspicuum, M. cookii, and M. asiaticum.

The intermediate-growing group includes M. marinum and M. gordonae. These organisms are usually pigmented and require 7 to 10 days of incubation for mature growth. M. marinum has an optimal growth temperature of 30° C., whereas M. gordonae prefers 35° C. M. gordonae is seldom if ever pathogenic, except in severely immunocompromised hosts.

The rapid-growing group of organisms includes nonpigmented and pigmented species that produce mature growth on agar plates, usually within 7 to 10 days. Nonpigmented pathogenic species are mostly grouped within the M. fortuitum complex, which includes the M. fortuitum group (M. fortuitum, M. peregrinum, and M. fortuitum third biovariant complex) and the M. chelonae-abscessus group (M. chelonae, formerly M. chelonae subspecies chelonae, M. abscessus, formerly M. chelonae subspecies abscessus, and M. mucogenicum, formerly M. chelonae-like organism). M. smegmatis may be pigmented or nonpigmented.

In certain embodiments, the mycobacteria is drug resistant. A “drug resistant” M. tuberculosis infection refers to a M. tuberculosis infection wherein the infecting strain is not held static or killed (is resistant to) one or more of so-called “front-line” chemotherapeutic agents effective in treating a M. tuberculosis infection (e.g., isoniazid, rifampin, ethambutol, streptomycin and pyrazinamide).

In certain embodiments, the mycobacteria is multi-drug resistant A “multi-drug resistant” M. tuberculosis infection refers to a M. tuberculosis infection wherein the infecting strain is resistant to two or more of “front-line” chemotherapeutic agents effective in treating a M. tuberculosis infection.

In certain embodiments, the mycobacteria is resistant against one or more drugs selected from isoniazid, rifampin, pyrazinamide and ethambutol. In certain embodiments, the mycobacteria is isoniazid-resistant M. tuberculosis.

The enantiomerically pure (R)-chloroquine agent may provide for one or more desirable properties over the corresponding enantiomerically pure (S)-chloroquine agent or racemic chloroquine agent, e.g., improved bioavailability, longer half-life, less frequent dosing, increased potency, etc, as described herein.

### Chloroquine Agent

As summarized above, chloroquine active agents find use in the subject methods. As used herein, the term “chloroquine active agent” refers to chloroquine or an analogue or derivative thereof.

As used herein, an enantiomerically pure chloroquine agent is one enantiomer of the agent that is substantially free from the other enantiomer of the agent (i.e., in enantiomeric excess). The enantiomerically chloroquine agent may be either the R or S enantiomer. As used herein, an enantiomerically pure (R)-chloroquine agent is substantially free from (S)-chloroquine agent (i.e., in enantiomeric excess). In other words, the “(R)” form of the chloroquine agent is substantially free from the “(S)” form of the compound and is, thus, in enantiomeric excess of the “(S)” form. As such, when the chloroquine agent is chloroquine, the (S)-chloroquine (S-CQ) and (R)-chloroquine (R-CQ) have the following structures, respectively.

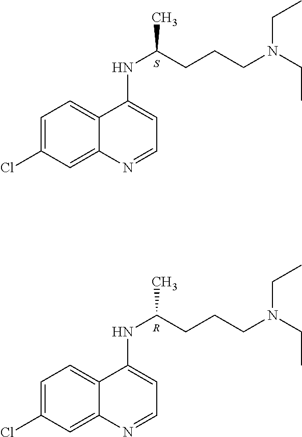

In some embodiments, (R)-chloroquine may also be referred to as (−)-chloroquine, and (S)-chloroquine may be referred to as (+)-chloroquine.

It is understood, when the chloroquine agent is not chloroquine, but is a chloroquine analog or derivative thereof (e.g., as described herein), that the (R) and (S) designations of the agent refer to configurations of the stereocenter located at the position alpha to the amino-quinoline core of the chloroquine agent, i.e., the sterocenter designated ‘*’ in the following general formula (I) of the chloroquine agent:

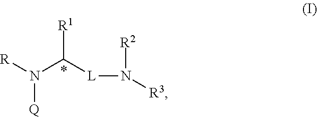

where Q is a quinoline-containing group and R and R¹-R³ are described herein.

One of ordinary skill in the art can readily determine which stereochemical configurations of the chloroquine agents find use in the subject method by reference of the structure of the chloroquine agent of interest to the analogous R-CQ structure shown above.

The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, or more than 97% by weight of the enantiomer.

As used herein and unless otherwise indicated, the term “enantiomerically pure (R)-chloroquine” refers to at least about 75% by weight (R)-chloroquine and at most about 25% by weight (S)-chloroquine, at least about 80% by weight (R)-chloroquine and at most about 20% by weight the (S)-enantiomer, at least about 90% by weight (R)-chloroquine and at most about 10% by weight the (S)-enantiomer, at least about 95% by weight (R)-chloroquine and at most about 5% by weight the (S)-enantiomer, or at least about 97% by weight (R)-chloroquine and at most about 3% by weight (S)-enantiomer.

In some embodiments, the chloroquine agent is described by Formula (I):

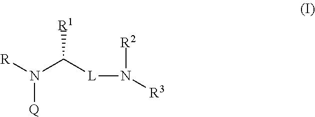

wherein:

Q is an optionally substituted quinoline-containing group;

R¹ is alkyl, alkenyl, alkynyl, aryl, aralkyl, aralkenyl, cycloalkyl, cycloalkenyl, heterocyclyl, heterocyclylalkyl, heterocyclylalkenyl, heteroaryl, heteroaralkyl, carboxyalkyl or carboxamidoalkyl;

R, R² and R³ are independently H, alkyl, alkenyl, alkynyl, aryl, aralkyl, aralkenyl, cycloalkyl, cycloalkenyl, heterocyclyl, heterocyclylalkyl, heterocyclylalkenyl, heteroaryl, heteroaralkyl, carboxyalkyl or carboxamidoalkyl, wherein R² and R³ may be cyclically linked such that they form a 4-8 membered N-containing heterocycle; and

L is a linker.

Any convenient quinoline-containing groups (Q) may be utilized in the subject agents. A quinoline-containing group is a group including at least one amino substituent and a heteroaryl group that includes a core amino-quinoline ring system. Quinoline-containing groups of interest include, but are not limited to, an 8-aminoquinoline, a 4-aminoquinoline, and an acridine such as a 9-aminoacridine.

In certain embodiments of formula (I), Q is selected from 8-amino-6-methoxyquinoline, 4-amino-7-chloroquine and 9-amino-6-chloro-2-methoxy-acridine, where Q may be optionally substituted.

Any convenient linkers may be utilized in the subject chloroquine agents. As used herein, the term “linker” or “linkage” refers to a linking moiety that connects two groups and has a backbone of 20 atoms or less in length. A linker or linkage may be a covalent bond that connects two groups or a chain of between 1 and 20 atoms in length, for example of about 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo(ethylene glycol); ethers, thioethers, tertiary amines, alkyls, which may be straight or branched, e.g., methyl, ethyl, n-propyl, 1-methylethyl(iso-propyl), n-butyl, n-pentyl, 1,1-dimethylethyl(t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

In some embodiments of formula (I), the linker is an alkyl group, such as a C₁-C₆ linking group. In certain embodiments of formula (I), the linker is a C₃ linking group.

In some embodiments, a chloroquine agent is described by Formula (II):

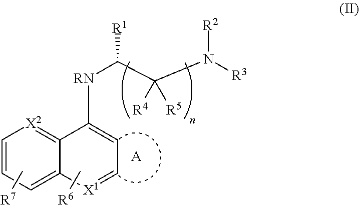

wherein:

X¹ is C—R⁶ or N and X² is C—R⁷ or N, wherein one of X¹ and X² is N;

R⁶ and R⁷ are independently 1-4 optional aryl substituents;

A is an optional fused benzo ring, optionally substituted with 1-4 aryl substituents;

R¹ is alkyl, alkenyl, alkynyl, aryl, aralkyl, aralkenyl, cycloalkyl, cycloalkenyl, heterocyclyl, heterocyclylalkyl, heterocyclylalkenyl, heteroaryl, heteroaralkyl, carboxyalkyl or carboxamidoalkyl;

R, R², R³, each R⁴ and each R⁵ are independently H, alkyl, alkenyl, alkynyl, aryl, aralkyl, aralkenyl, cycloalkyl, cycloalkenyl, heterocyclyl, heterocyclylalkyl, heterocyclylalkenyl, heteroaryl, heteroaralkyl, carboxyalkyl or carboxamidoalkyl, wherein R² and R³ may be cyclically linked such that they form a 4-8 membered N-containing heterocycle; and

n is 1-5.

In certain embodiments of formula (II), X¹ is N and X² is C—R⁷. In certain embodiments of formula (II), X² is N and X¹ is C—R⁶.

In certain embodiments of formula (II), each R⁴ and each R⁵ is H.

In certain embodiments of formula (II), n is 1. In certain embodiments of formula (II), n is 2. In certain embodiments of formula (II), n is 3. In certain embodiments of formula (II), n is 4. In certain embodiments of formula (II), n is 5.

In certain embodiments of formula (II), n is 3 and R⁴ and R⁵ are each H.

In some embodiments of formula (II), R is H or a lower alkyl. In certain embodiments of formula (II), R is H. In certain embodiments of formula (II), R is a lower alkyl (e.g., methyl).

In some embodiments of formula (II), R¹ is a lower alkyl. In certain embodiments of formula (II), R¹ is methyl, ethyl, propyl, isopropyl, butyl, iso-butyl or tert butyl. In certain embodiments of formula (II), R¹ is methyl.

In some embodiments of formula (II), R² and R³ are independently a lower alkyl. In certain embodiments of formula (II), R² and R³ are independently methyl, ethyl, propyl, isopropyl, butyl, iso-butyl or tert butyl. In certain embodiments of formula (II), R² and R³ are each ethyl. In certain embodiments of formula (II), R² and R³ are each hydrogen.

In some embodiments of formula (II), each R⁶ and each R⁷ are independently selected from the group consisting of alkyl, aryl, aralkyl, alkoxy, aryloxy, aralkoxy, cycloalkyl, cycloalkoxy, heterocyclyl, heterocyclyloxy, heterocyclylalkyl, heteroaryl, heteroaralkyl, keto, hydroxy, thio, amino, alkylamino, alkanoylamino, aroylamino, aralkanoylamino, carboxy, carbonate, carbamate, guanidinyl, urea, halo, cyano, nitro, formyl, acyl, phosphoryl, sulfonyl and sulfonamido and is optionally substituted with 1-3 J groups, wherein J is alkyl, aryl, aralkyl, alkoxy, aryloxy, heterocyclyl, heterocyclyloxy, keto, hydroxy, thio, amino, alkanoylamino, aroylamino, carboxy, carbonate, carbamate, guanidinyl, urea, halo, cyano, nitro, formyl, acyl, phosphoryl, sulfonyl, or sulfonamide.

In some embodiments of formula (II), A is present. In some embodiments of formula (II), A is absent.

In some embodiments, a chloroquine agent is described by one of Formulae (III), (IV) and (V):

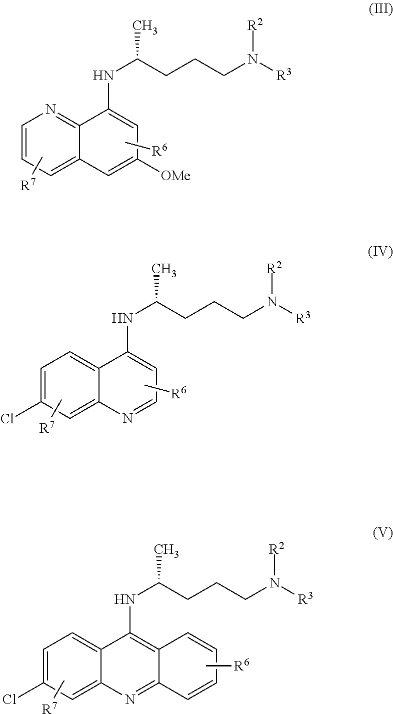

wherein:

R⁶ and R⁷ are independently 1-5 optional aryl substituents;

R² and R³ are independently H, alkyl, alkenyl, alkynyl, aryl, aralkyl, aralkenyl, cycloalkyl, cycloalkenyl, heterocyclyl, heterocyclylalkyl, heterocyclylalkenyl, heteroaryl, heteroaralkyl, carboxyalkyl or carboxamidoalkyl, wherein R² and R³ may be cyclically linked such that they form a 4-8 membered N-containing heterocycle.

In some embodiments, a chloroquine agent is described by Formula (III). In some embodiments, a chloroquine agent is described by Formula (IV). In some embodiments, a chloroquine agent is described by Formula (V).

In some embodiments of formulae (III), (IV) and (IV), R² and R³ are independently a lower alkyl. In certain embodiments of formulae (III), (IV) and (IV), R² and R³ are independently methyl, ethyl, propyl, isopropyl, butyl, iso-butyl or tert butyl. In certain embodiments of formulae (III), (IV) and (IV), R² and R³ are each ethyl. In certain embodiments of formulae (III), (IV) and (IV), R² and R³ are each hydrogen.

In some embodiments of formulae (III), (IV) and (IV), each R⁶ and each R⁷ are independently selected from the group consisting of alkyl, aryl, aralkyl, alkoxy, aryloxy, aralkoxy, cycloalkyl, cycloalkoxy, heterocyclyl, heterocyclyloxy, heterocyclylalkyl, heteroaryl, heteroaralkyl, keto, hydroxy, thio, amino, alkylamino, alkanoylamino, aroylamino, aralkanoylamino, carboxy, carbonate, carbamate, guanidinyl, urea, halo, cyano, nitro, formyl, acyl, phosphoryl, sulfonyl and sulfonamido and is optionally substituted with 1-3 J groups, wherein J is alkyl, aryl, aralkyl, alkoxy, aryloxy, heterocyclyl, heterocyclyloxy, keto, hydroxy, thio, amino, alkanoylamino, aroylamino, carboxy, carbonate, carbamate, guanidinyl, urea, halo, cyano, nitro, formyl, acyl, phosphoryl, sulfonyl, or sulfonamide.

In some embodiments of formulae (III), (IV) and (IV), R⁶ is H.

In some embodiments of formulae (III), (IV) and (IV), R⁷ is H.

Chloroquine active agents which find use in the subject methods include, but are not limited to, primaquine, pamaquine, tafenoquine, mepacrine, chloroquine, desethylchloroquine.

In some embodiments, the (R)-chloroquine agent has the following structure:

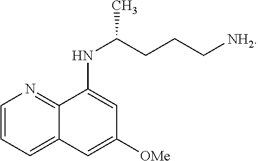 [SMILES: COc1cc(N[C@H](C)CCCN)c2ncccc2c1]

In some embodiments, the (R)-chloroquine agent has the following structure:

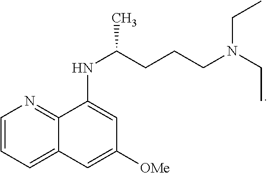 [SMILES: CCN(CC)CCC[C@@H](C)Nc1cc(OC)cc2cccnc12]

In some embodiments, the (R)-chloroquine agent has the following structure:

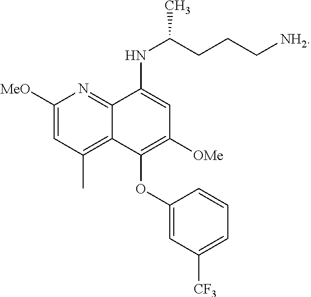 [SMILES: COc1cc(C)c2c(Oc3cccc(C(F)(F)F)c3)c(OC)cc(N[C@H](C)CCCN)c2n1]

In some embodiments, the (R)-chloroquine agent has the following structure:

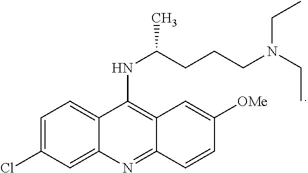 [SMILES: CCN(CC)CCC[C@@H](C)Nc1c2ccc(Cl)cc2nc2ccc(OC)cc12]

In some embodiments, the (R)-chloroquine agent has the following structure:

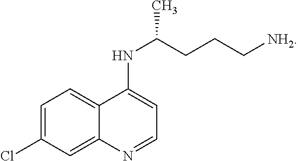 [SMILES: C[C@H](CCCN)Nc1ccnc2cc(Cl)ccc12]

The subject chloroquine agents contain asymmetric carbon atoms and thus exist in different stereoisomeric forms. The chloroquine agents may be provided in at least partially purified stereochemical forms or as mixtures of enantiomers and/or diastereomers. Unless a specific stereochemistry is indicated, it should be understood that all of the optical isomers and mixtures thereof are encompassed. In some embodiments, the chloroquine agents are provided in at least partially purified stereochemical form. An enantiomeric excess or a diasterieomeric excess exists where one enantiomer or diasteriomer, respectively, is present in larger chemical amounts than another. As used herein, the “enantiomeric ratio” is the molar ratio between two enantiomeric structures in a mixture. The “diasteriomeric ratio” is the molar ratio between two specified diasteriomeric structures in a mixture. For enantiomeric or diasteriomeric epoxide compounds of the instant invention, the enantiomeric or diasteriomeric ratio is the molar ratio between those two stereoisomers in a mixture, e.g. the ratio between an (R) and an (S) stereoisomer. In the case of compounds that are present in at least partially purified stereochemical form, the enantiomeric ratio or diasteriomeric ratio is in some embodiments at least 2. In other embodiments of the invention, the enantiomeric ratio or diasteriomeric ratio is at least 4, at least 8, at least 20, at least 50, or even higher.

When a particular stereochemical or geometric isomer is specified in a structure, or when a particular isomeric purity is indicated, the particular form can be obtained by asymmetric synthesis, synthesis from optically pure precursors, or by resolution of racemates or other mixtures of stereochemical or geometric isomers. Resolution of racemates or other mixtures may also be accomplished, for example, by conventional methods such as crystallization in the presence of a resolving agent, or chromatography, using, for example a chiral HPLC column. In some embodiments, the compounds of the invention are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention, which, upon administration to a recipient, is capable of providing or provides (directly or indirectly) a compound of the invention.

In some embodiments, the chloroquine agents are provided in the form of pharmaceutically acceptable salts. Compounds containing an amine may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

### Methods

As summarized above, aspects of the method include administering to a subject in need thereof a therapeutically effective amount of an enantiomerically pure (R)-chloroquine agent effective to treat or prevent TB, or other mycobacteria infection, in the subject. By “an amount effective to treat or prevent TB, or other mycobacteria infection” is meant the concentration of a compound that is sufficient to elicit the desired biological effect (e.g., treatment or prevention of primary or secondary tuberculosis, or the atypical mycobacteria infection).

As used herein, the term “primary tuberculosis” refers to clinical illness (manifestation of disease symptoms) directly following infection with M. tuberculosis. See, Harrison's Principles of Internal Medicine, Chapter 150, pp. 953-966 (16th ed., Braunwald, et al., eds., 2005). As used herein, the tem “secondary tuberculosis” or “postprimary tuberculosis” refers to the reactivation of a dormant, inactive or latent M. tuberculosis infection. See, Harrison's Principles of Internal Medicine, supra. An “active infection of M. tuberculosis” refers to a M. tuberculosis infection with manifested disease symptoms. An “inactive, dormant or latent infection of M. tuberculosis” refers to a M. tuberculosis infection without manifested disease symptoms.

The term “tuberculosis reactivation” refers to the later manifestation of disease symptoms in an individual that tests positive in a tuberculin test but does not have apparent disease symptoms. The individual is infected with M. tuberculosis, and may or may not have previously manifested active disease symptoms that had been treated sufficiently to bring the tuberculosis into an inactive or latent state. Methods for the prevention or treatment of tuberculosis reactivation can be initiated in an individual manifesting active symptoms of disease, however.

As used herein, the term “subject” refers to a mammal. Exemplary mammals include, but are not limited to, humans, domestic animals (e.g., a dog, cat, or the like), farm animals (e.g., a cow, a sheep, a pig, a horse, or the like) or laboratory animals (e.g., a monkey, a rat, a mouse, a rabbit, a guinea pig, or the like). In certain embodiments, the subject is human.

As used herein, the terms “treatment,” “treating,” and the like, refer to obtaining a desired pharmacologic and/or physiologic effect. The effect may be prophylactic in terms of completely or partially preventing a disease (e.g., mycobacterial infection) or symptom thereof and/or may be therapeutic in terms of a partial or complete cure for a disease (e.g., mycobacterial infection) and/or adverse effect attributable to the disease (e.g., mycobacterial infection). As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance. “Treatment,” as used herein, covers any treatment of a disease in a mammal, particularly in a human, and includes: (a) preventing the disease (e.g., mycobacterial infection) from occurring in a subject which may be predisposed to the disease (e.g., mycobacterial infection) but has not yet been diagnosed as having it; (b) inhibiting the disease (e.g., mycobacterial infection), i.e., arresting its development; and (c) relieving the disease (e.g., TB infection), i.e., causing regression of the disease (e.g., mycobacterial infection).

In some cases, a subject method involves administering to an individual in need thereof an effective amount of an enantiomerically pure (R)-chloroquine agent. In some embodiments, an “effective amount” is an amount that, when administered to an individual in one or more doses, in monotherapy or in combination therapy, is effective to reduce bacterial load in the individual by at least about 20% (20% suppression), at least about 30% (30% suppression), at least about 40% (40% suppression), at least about 50% (50% suppression), at least about 60% (60% suppression), at least about 70% (70% suppression), at least about 80% (80% suppression), or at least about 90% (90% suppression), compared to the bacterial load in the individual in the absence of treatment with the enantiomerically pure (R)-chloroquine agent, or alternatively, compared to the bacterial load in the individual after treatment with the corresponding enantiomerically pure (S)-chloroquine agent, or racemic chloroquine agent.

In some embodiments, an “effective amount” of an enantiomerically pure (R)-chloroquine agent is an amount that, when administered in one or more doses to an individual having a mycobacterial infection, is effective to achieve a 1.5-log, a 2-log, a 2.5-log, a 3-log, a 3.5-log, a 4-log, a 4.5-log, or a 5-log reduction in mycobacteria colony formation unit in the serum of the individual.

In some embodiments, an effective amount of an enantiomerically pure (R)-chloroquine agent is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of an enantiomerically pure (R)-chloroquine agent is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of an enantiomerically pure (R)-chloroquine agent is administered. In other embodiments, multiple doses of an active agent are administered. Where multiple doses are administered over a period of time, enantiomerically pure (R)-chloroquine agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an enantiomerically pure (R)-chloroquine agent is administered at any of the aforementioned frequencies for one week, two weeks, one month, two months, six months, one year, or two years, or more, depending on various factors.

Administration of an effective amount of an enantiomerically pure (R)-chloroquine agent to an individual in need thereof can result in one or more of: 1) a reduction in bacterial load; 2) a reduction in bacterial load in a target biological sample; 3) a reduction in the spread of a mycobacteria from one cell to another cell in an individual; 4) a reduction in mycobacterial entry into (e.g., reduction of internalization of a mycobacteria into) a cell; 5) a reduction in time to seroconversion (mycobacteria undetectable in patient serum); 6) an increase in the rate of sustained response to therapy; 7) a reduction of morbidity or mortality in clinical outcomes; 8) shortening the total length of treatment when combined with other anti-TB agents; and 9) an improvement in an indicator of disease response (e.g., a reduction in one or more symptoms of a bacterial infection, such as fever, etc.).

Any of a variety of methods can be used to determine whether a treatment method is effective. For example, a biological sample obtained from an individual who has been treated with a subject method can be assayed for the presence and/or level of a bacteria-encoded protein, for the presence and/or level of mycobacteria genomes, and the like. Methods of measuring mycobacteria concentration will be known to one of ordinary skill in the art and may include: plaque assay (determine the number of colony forming units (CFU) in a sample by plating a known volume and/or dilution of sample on agar plates and count the number of formed bacterial colonies); fluorescent focus assay (FFA); protein bases assays (e.g., hemagglutination assay, bicinchoninic acid assay, single radial immunodiffusion assay, and the like); transmission Electron Microscopy (TEM); flow cytometry (e.g., using antibodies and/or probes against viral specific proteins and/or nucleic acids); etc.

In some embodiments, the subject methods further include administering one or more additional agents, e.g., antibacterial agents such as anti-TB agents.

In certain embodiments, the subject methods include administering a therapeutically effective amount of one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic and streptomycin. In certain embodiments, the subject methods include administering a therapeutically effective amount of one or more additional agents selected from isoniazid, pyrazinamide and rifampin.

In certain embodiments, the enantiomerically pure (R)-chloroquine active agent and the one or more additional agents are administered at the same time. In certain embodiments, the enantiomerically pure (R)-chloroquine active agent and the one or more additional agents are administered as separate formulations.

In certain embodiments, the enantiomerically pure (R)-chloroquine active agent and the one or more additional agents are administered in a single formulation. In certain embodiments, the enantiomerically pure (R)-chloroquine active agent and the one or more additional agents are administered sequentially.

Combination therapy includes administration of a single pharmaceutical dosage formulation which contains the subject compound and one or more additional agents; as well as administration of the subject compound and one or more additional agent(s) in its own separate pharmaceutical dosage formulation. For example, a subject compound and an additional agent active against an infectious disease (e.g., a bacterial infection) can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially.

### Methods of Inhibiting TB

Also provided are methods of inhibiting TB in a cell. In some embodiments, the cell is in vitro. In other embodiments, the cell is in vivo.

In some embodiments, the method includes contacting a sample comprising a cell with an enantiomerically pure chloroquine agent (e.g., as described herein). Any convenient methods of contacting a sample with the agent may be utilized.

The cell may in a biological sample. The term “sample” as used herein relates to a material or mixture of materials, typically, although not necessarily, in liquid form, containing one or more analytes of interest. In one embodiment, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In certain embodiments, the sample has been removed from an animal or plant. Biological samples of the invention include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

In some embodiments of the method, the enantiomerically pure (R)-chloroquine agent inhibits TB replication in a cell. In some cases, the cell is infected with a drug-resistant strain or a multi-drug resistant strain of TB. In certain embodiments of the method, the enantiomerically pure (R)-chloroquine agent inhibits TB replication in the cell with an EC₈₀ of about 10 μM or less, such as about 3 μM or less, 1 μM or less, 300 nM or less, or 100 nM or less, 30 nM or less or even 10 nM or less.

In certain embodiments of the method, the enantiomerically pure (R)-chloroquine agent inhibits TB replication in the cell and has an EC₈₀ that is 3-fold less than that of the corresponding enantiomerically pure (S)-chloroquine agent, such as 4-fold less, 5-fold less, 10-fold less, 20-fold less, 50-fold less, or 100-fold less than that of the corresponding enantiomerically pure (S)-chloroquine agent. For example, in certain embodiments, the enantiomerically pure (R)-chloroquine agent inhibits TB replication in the cell with an EC₈₀ of about luM or less while the corresponding enantiomerically pure (S)-chloroquine agent inhibits TB replication in the cell with an EC₈₀ of about 10 μM or more, such as about 20 μM or more, about 50 μM or more, or about 100 μM or more.

In certain embodiments of the method, the enantiomerically pure (R)-chloroquine agent inhibits TB replication in the cell and has an EC₈₀ that is 3-fold less than that of the corresponding racemic chloroquine agent, such as 4-fold less, 5-fold less, 10-fold less, 20-fold less, 50-fold less, or 100-fold less than that of the corresponding racemic chloroquine agent. For example, in certain embodiments, the enantiomerically pure (R)-chloroquine agent inhibits TB replication in the cell with an EC₈₀ of about 1 uM or less while the corresponding racemic chloroquine agent inhibits TB replication in the cell with an EC₈₀ of about 10 μM or more, such as about 20 μM or more, about 50 μM or more, or about 100 μM or more.

In some embodiments of the method, the enantiomerically pure (R)-chloroquine agent has a microbicidal activity in a cell infected with TB. In some cases, the cell is infected with a drug-resistant strain or a multi-drug resistant strain of TB. In certain embodiments, the cell is infected with an isoniazid-resistant strain of TB. The microbicidal activity or potency of the agents may be assayed using any convenient methods, e.g., using a cell based antibacterial assay.

In certain embodiments, the enantiomerically pure (R)-chloroquine agent has a microbicidal activity and is about 3-fold or more potent that the corresponding enantiomerically pure (S)-chloroquine agent, such as about 10-fold or more potent, about 30-fold or more potent, about 100-fold or more potent, about 300-fold or more potent, or even about 1000-fold or more potent.

In certain embodiments, the enantiomerically pure (R)-chloroquine agent has a microbicidal activity and is about 3-fold or more potent that the corresponding racemic chloroquine agent, such as about 10-fold or more potent, about 30-fold or more potent, about 100-fold or more potent, about 300-fold or more potent, or even about 1000-fold or more potent.

### Pharmaceutical Compositions

The compounds of the invention may be administered as a pharmaceutical composition containing, for example, a subject compound (e.g., as described herein) and a pharmaceutically acceptable carrier.

The pharmaceutical composition may include a therapeutically effective amount of the subject compound. The term “therapeutically effective amount” is used to indicate an amount of an active compound, or pharmaceutical agent that elicits a biological or medicinal response.

In some embodiments, the pharmaceutical composition is a composition for treating an individual infected with a mycobacteria of the M. tuberculosis complex, comprising a therapeutically effective amount of an enantiomerically pure (R)-chloroquine active agent or a pharmaceutically acceptable salt thereof.

In certain embodiments, the pharmaceutical composition further comprises a therapeutically effective amount of one or more additional agents selected from the group consisting of: isoniazid, rifampin, pyrazinamide and ethambutol, rifabutin, rifapentine, amikacin, capremycin, cycloserine, ethionamide, levofloxacin, moxifloxacin, para-aminosalicylic, streptomycin, nitazoxanide, gleevec and CPZEN-45.

In certain embodiments, the pharmaceutical composition further comprises a therapeutically effective amount of one or more additional agents selected from isoniazid, pyrazinamide and rifampin.

Pharmaceutical compositions include a compound (either alone or in the presence of one or more additional active agents) present in a pharmaceutically acceptable vehicle. The term “pharmaceutically acceptable” means approved by a regulatory agency of the Federal or a state government or listed in the U.S. Pharmacopeia or other generally recognized pharmacopeia for use in mammals, such as humans. The term “vehicle” or “carrier” are used interchangeably and refer to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the invention and pharmaceutically acceptable vehicles, excipients, or diluents may be sterile. In some instances, an aqueous medium is employed as a vehicle when the compound of the invention is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular compound, as well as by the particular method used to administer the composition. Accordingly, there is a wide variety of suitable formulations of the pharmaceutical composition of the present invention.

Administration of compounds of the invention may be systemic or local. In certain embodiments administration to a mammal will result in systemic release of a compound of the invention (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the compounds and compositions of the invention are administered orally. In certain embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

A compound of the invention may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. A compound of the invention may also be incorporated into existing nutraceutical formulations, such as are available conventionally, which may also include an herbal extract.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present invention should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the compounds may be administered in the form of a free base, their pharmaceutically acceptable salts, or they may also be used alone or in appropriate association, as well as in combination, with other pharmaceutically active compounds.

### Kits

Also provided are kits that include compounds of the invention. Kits of the invention may include one or more dosages of the compound, and optionally one or more dosages of one or more additional active agents (e.g., anti-TB agents). Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to treat infectious disease such as a TB.

In some embodiments, the kit includes an enantiomerically pure (R)-chloroquine agent (e.g., as described herein) and at least one additional compound is selected from isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin. In certain embodiments, the kit includes one or more additional compounds selected from isoniazid, pyrazinamide and rifampin.

In addition to above-mentioned components, the subject kits may further include instructions for using the components of the kit to practice the subject methods, i.e., to provide instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

The compounds, compositions and methods of the invention, e.g., as described above, find use in a variety of applications. Applications of interest include, but are not limited to: therapeutic applications, research applications, and diagnostic applications. Each of these different applications are now reviewed in greater details below.

The subject compounds find use in a variety of therapeutic applications. Therapeutic and diagnostic applications of interest include those applications in which diagnosis and/or treatment of TB, or other atypical mycobacteria, are of interest.

Accordingly, the methods comprise administering to the mammalian host in need thereof a pharmaceutical composition as described above. As such, the pharmaceutical compositions of the invention are used in methods for treating or preventing particular diseases, e.g., TB. The methods comprise, for example, administering to the mammalian host in need thereof a therapeutically-effective amount of a pharmaceutical composition as described above.

In some embodiments, the subject compounds may be administered in combination with one or more additional compounds or therapies, including a second target-binding molecule, an antibacterial agent, surgery, catheter devices, and radiation. Combination therapy includes administration of a single pharmaceutical dosage formulation which contains the subject compound and one or more additional agents; as well as administration of the subject compound and one or more additional agent(s) in its own separate pharmaceutical dosage formulation. For example, a subject compound and an additional agent active against an infectious disease (e.g., a bacterial infection) can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially.

The subject compounds and methods find use in a variety of research applications. The subject compounds and methods may be used to analyze the roles of chloroquine agents in modulating various biological processes involved with TB.

### EXAMPLES

### Example 1

### Preparation of Compounds

R-chloroquine (R-CQ) and S-chloroquine (S-CQ) were synthesized by resolving the racemic diethyl-pentane-1,4-diamine into enantiomers and then condensing each enantiomer with the 4-amino-7-chloroquinoline core.

### Example 2

### Inhibition of TB

### General Method:

The following general method may be adapted for use in assaying compounds of interest.

The virulent Mtb strain H37Rv or the INH-resistant ΔkatG, where the katG locus is removed (which confers resistance to INH), is grown to midlog phase in Middlebrook 7H9 medium. RAW 264.7 cells (ATCC) are maintained in complete DMEM (10% FBS, 1% P/S, 1% NEAA, 1% L-Glut). The day before infection, RAW 264.7 cells are plated into 24-well plate at 200,000 cells/well in complete media with no P/S. The next day, the media is replaced with fresh media and 1,000,000 Mtb (MOI of 5) is added to the well with varying concentrations of the indicated drugs. 4 hours later, bacteria is removed, and the cells are washed with PBS and replenished with fresh media containing drugs. 72 hours later, media is removed; cells are lysed and plated on agar plates for 3 weeks and the CFU is counted.

### Results:

A. R-CQ Inhibits TB Replication in Macrophages More than S-CQ or CQ

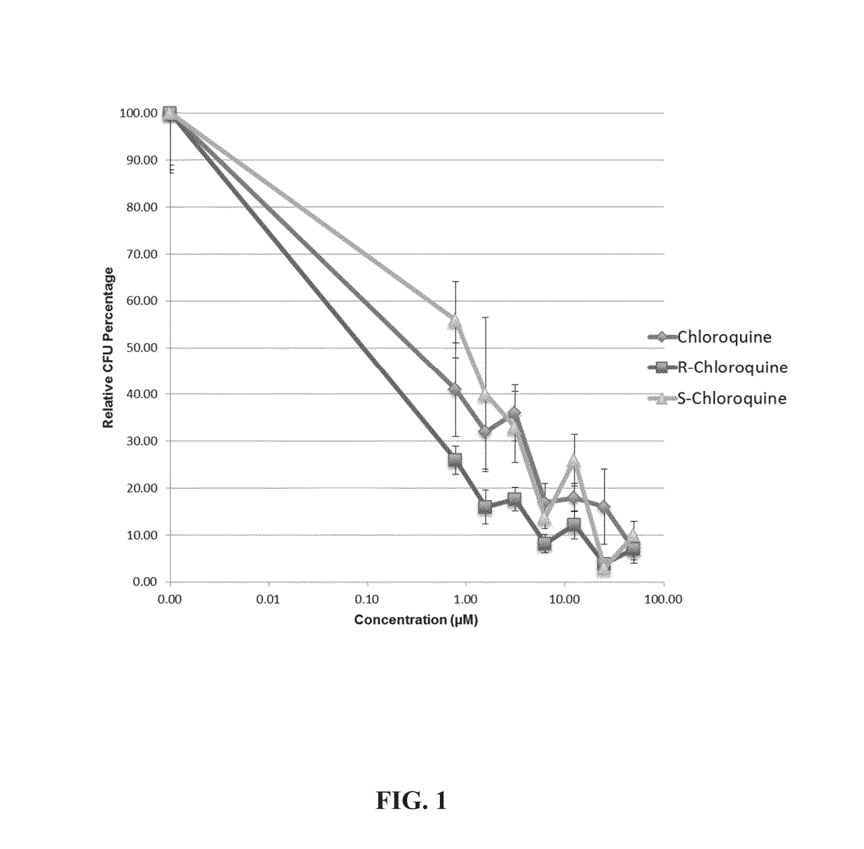

R-CQ, S-CQ and racemic CQ were assayed at the following concentrations: 50; 25; 12.5; 6.25; 3.125; 1.5625; and 0.78125 μM and water-treated control (see FIG. 1). No cellular toxicity is observed at these concentrations of the compounds. The microbicidal activity of R-CQ is significantly higher than either S-CQ or racemic CQ where R-CQ has an EC₈₀ of 1 μM while S-CQ and racemic CQ have an EC₈₀ of ˜10 μM.

B. R-CQ is More Active than S-CQ or CQ in INH-Resistant TB, katG Knock-Out

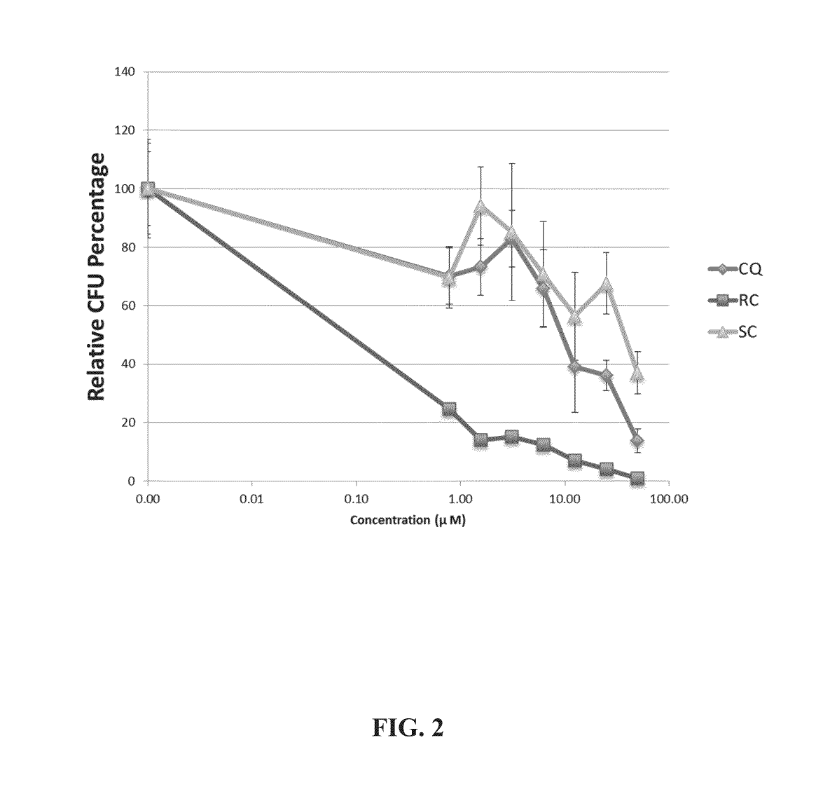

R-CQ, S-CQ and racemic CQ were assayed at the following concentrations: 50; 25; 12.5; 6.25; 3.125; 1.5625; and 0.78125 μM and water-treated control (see FIG. 2). No cellular toxicity is observed at these concentrations of the compounds. R-CQ also exerts significantly higher microbicidal capacity against the INH-resistant ΔkatG H37Rv than either S-CQ or racemic CQ. The EC80 of R-CQ against ΔkatG H37Rv is 1 μM while S-CQ achieves only 60% inhibition at 50 μM (the highest concentration tested), and racemic CQ has an EC₈₀ of ˜50 μM.

### Example 3

### In Vitro Evaluation of Anti-Nontuberculous Mycobacteria (NTM) Activity

Minimal Inhibitory Concentration (MIC) and Minimal bactericidal activity (MBC) testing is performed by microbroth dilution using general methods (e.g., as described herein) and includes one or more of the NTM priority organisms listed below:

M. avium (two strains)

M. intracellulare

M. abscessus ssp abscessus (two strains)

M. abscessus ssp massiliense

M. kansasii

M. chelonae

M. xenopi

M. ulcerans

### Example 4

### In Vivo Testing of Anti-Nontuberculosis Mycobacteria Activity

### Determination of Maximum Tolerated Dose (MTD)

C57BL/6 female mice (6-8 weeks in age) are administered a one-time dose (oral gavage) for 3 days of the compound of interest at concentrations of 30, 100, or 300 mg/Kg. The compounds are dissolved in an appropriate vehicle (EtOH, DMSO or methylcellulose), administered in a solution if necessary. There are 3 animals per dose and they are observed post-administration for 4 hours, again 6 hours later and then twice daily for the duration of the study (1 week). If an animal exhibits obvious signs of distress (hunched posture, ruffled fur, etc.), it is euthanized. The MTD (mg/Kg) is the highest dose that results in no lethality/tissue abnormality.

In Vivo Efficacy Mouse Models for M. avium and M. intracellulare

The primary model is the Beige-C57BL/J bg model which expresses a Th2 immune profile and increased susceptibility to infection. The Gamma Interferon Knock Out (GKO) and the C3HeB/FeJ models for efficacy testing of compounds against M. avium and M. intracellulare are also used.

Mice are infected intravenously (IV) with 3×10⁷ cfu or high dose aerosol (HDA) of 1×10⁸ cfu of M. avium or M. intracellulare. In the Acute model after seven days, therapy is initiated and continued for ten days (day 8-18). On day 1, 7 and 19 the mice are harvested. Lung, spleen and liver are aseptically dissected, weighed, gross histological evaluation completed and homogenized. Serial dilutions are plated onto 7H11 agar for quantitative culture. In the chronic model after 7 days, therapy is initiated and continued for four weeks. The mice are then harvested on day 1, 14 and 29. Lung, spleen and liver are aseptically dissected, weighed, gross histological evaluation completed and homogenized. Serial dilutions are plated onto 7H11 agar for quantitative culture.

In Vivo Efficacy Mouse Models for M. Abscessus, M. Massiliense, M. kansasii, M. chelonae and M. xenopi

Efficacy testing of compounds of interest against M. abscessus, M. massiliense, M. kansasii, M. chelonae and M. xenopi is performed. The primary model is the SCID model. An additional model for efficacy testing of compounds against NTMs is the GMCSF−/− and cystic fibrosis B6CFTRtm1UNC/CFTRtm1UNC.

Mice are infected intravenously (IV) or gavage with 1×10⁷ cfu of M. abscessus, M. kansasii, M. chelonae and M. xenopi. In the Acute model after seven days, therapy is initiated and continued for ten days (day 8-18). On day 1, 7 and 19 the mice are harvested. Lung, spleen and liver are aseptically dissected, weighed, gross histological evaluation completed and homogenized. Serial dilutions are plated onto 7H11 agar for quantitative culture.
In the chronic model after 7 days, therapy is initiated and continued for four weeks. The mice are harvested on day 1, 14 and 29. Lung, spleen and liver are aseptically dissected, weighed, gross histological evaluation completed and homogenized. Serial dilutions are plated onto 7H11 agar for quantitative culture.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the embodiments shown and described herein. Rather, the scope and spirit of present invention is embodied by the appended embodiments.

### Embodiments

Aspects of the present disclosure include a method of treating a subject infected with a mycobacteria of the M. tuberculosis complex. In some embodiments, the method comprises administering to a subject in need thereof a therapeutically effective amount of an enantiomerically pure (R)-chloroquine agent. In some embodiments, the mycobacteria is selected from M. tuberculosis, M. paratuberculosis, M. avium, M. intracellulare, M. abscessus ssp abscessus, M. abscessus ssp massiliense, M. kansasii, M. chelonae, M. xenopi and M. leprae. In some embodiments, the mycobacteria is M. tuberculosis. In some embodiments, the mycobacteria is drug resistant. In some embodiments, the mycobacteria is resistant against one or more drugs selected from isoniazid, rifampin, pyrazinamide and ethambutol. In some embodiments, the mycobacteria is isoniazid-resistant M. tuberculosis. In some embodiments, the enantiomerically pure (R)-chloroquine active agent has increased efficacy relative to a racemic mixture of the agent. In some embodiments, the (R)-chloroquine active agent comprises an enantiomeric excess of 90% or more (e.g., 95% or more, 98% or more or 99% or more) of the (R)-enantiomer. In some embodiments, the method further comprises administering a therapeutically effective amount of one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin. In some embodiments, the enantiomerically pure (R)-chloroquine active agent and the one or more additional agents are administered at the same time. In some embodiments, enantiomerically pure (R)-chloroquine active agent and the one or more additional agents are administered as separate formulations. In some embodiments, the enantiomerically pure (R)-chloroquine active agent and the one or more additional agents are administered in a single formulation. In some embodiments, the enantiomerically pure (R)-chloroquine active agent and the one or more additional agents are administered sequentially. In some embodiments, the enantiomerically pure (R)-chloroquine active agent is administered to the subject one time a day. In some embodiments, the enantiomerically pure (R)-chloroquine active agent is administered to the subject one time a week. In some embodiments, the enantiomerically pure (R)-chloroquine active agent is administered to the subject one time a month. In some embodiments, the enantiomerically pure (R)-chloroquine active agent is administered orally. In some embodiments, the enantiomerically pure (R)-chloroquine active agent is administered via inhalation. In some embodiments, the subject is human. Aspects of the present disclosure include a pharmaceutical composition for treating an individual infected with a mycobacteria of the M. tuberculosis complex, comprising a therapeutically effective amount of an enantiomerically pure (R)-chloroquine active agent or a pharmaceutically acceptable salt thereof. In some embodiments, the pharmaceutical composition further comprises a therapeutically effective amount of one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin. Aspects of the present disclosure include a kit for treating an individual infected with a mycobacteria of the M. tuberculosis complex. In some embodiments, the kit includes a therapeutically effective amount of an enantiomerically pure (R)-chloroquine active agent; and one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin.

What is claimed is:

1. A method of treating a subject infected with a mycobacteria of the M. tuberculosis complex, the method comprising administering to a subject in need thereof a therapeutically effective amount of an enantiomerically pure (R)-chloroquine agent.

2. The method of claim 1, wherein the mycobacteria is selected from M. tuberculosis, M. paratuberculosis, M. avium, M. intracellulare, M. abscessus ssp abscessus, M. abscessus ssp massiliense, M. kansasii, M. chelonae, M. xenopi and M. leprae.

3. The method of claim 2, wherein the mycobacteria is M. tuberculosis.

4. The method of claim 3, wherein the mycobacteria is drug resistant.

5. The method of claim 3, wherein the mycobacteria is resistant against one or more drugs selected from isoniazid, rifampin, pyrazinamide and ethambutol.

6. The method of claim 5, wherein the mycobacteria is isoniazid-resistant M. tuberculosis.

7. The method of claim 1, wherein the enantiomerically pure (R)-chloroquine active agent has increased efficacy relative to a racemic mixture of the agent.

8. The method of claim 1, wherein the (R)-chloroquine active agent comprises an enantiomeric excess of 90% or more of the (R)-enantiomer.

9. The method of claim 1, further comprising administering a therapeutically effective amount of one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin.

10. The method of claim 9, wherein the enantiomerically pure (R)-chloroquine active agent and the one or more additional agents are administered at the same time.

11. The method of claim 9, enantiomerically pure (R)-chloroquine active agent and the one or more additional agents are administered as separate formulations.

12. The method of claim 9, wherein the enantiomerically pure (R)-chloroquine active agent and the one or more additional agents are administered in a single formulation.

13. The method of claim 9, wherein the enantiomerically pure (R)-chloroquine active agent and the one or more additional agents are administered sequentially.

14. The method of claim 1, wherein the enantiomerically pure (R)-chloroquine active agent is administered to the subject one time a day.

15. The method of claim 1, wherein the enantiomerically pure (R)-chloroquine active agent is administered to the subject one time a week.

16. The method of claim 1, wherein the enantiomerically pure (R)-chloroquine active agent is administered orally.

17. The method of claim 1, wherein the enantiomerically pure (R)-chloroquine active agent is administered via inhalation.

18. A pharmaceutical composition for treating an individual infected with a mycobacteria of the M. tuberculosis complex, comprising a therapeutically effective amount of an enantiomerically pure (R)-chloroquine active agent or a pharmaceutically acceptable salt thereof.

19. The pharmaceutical composition of claim 1, further comprising a therapeutically effective amount of one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin.

20. A kit for treating an individual infected with a mycobacteria of the M. tuberculosis complex, comprising:
a therapeutically effective amount of an enantiomerically pure (R)-chloroquine active agent; and
one or more additional agents selected from the group consisting of isoniazid, rifampin, pyrazinamide and ethambutol, rifabutin, rifapentine, amikacin, capremycin, cycloserine, ethionamide, levofloxacin, moxifloxacin, para-aminosalicylic, nitazoxanide, gleevec, CPZEN-45 and streptomycin.
